(19) United States
(12) Patent Application Publication
(10) Pub. No.: US 2009/0298745 A1
(43) Pub. Date: Dec. 3, 2009

(54) Treatment of Diabetes with Glycogen Phosphorylase Inhibitors

(76) Inventors: Gerard Hugh Thomas, Oxford (GB); Mikael Thomsen, Hvldovre (DK)

Correspondence Address: OSI PHARMACEUTICALS, INC., 41 PINELAWN ROAD, MELVILLE, NY, 11747, US

(21) Appl. No.: 11/792,186

(22) PCT Filed: Dec. 2, 2005
(86) PCT No.: PCT/GB2005/050232

§ 371 Date: Jul. 23, 2009

Related U.S. Application Data
(60) Provisional application No. 60/632,591, filed on Dec. 2, 2004.

(51) Int. Cl. A61K 38/28; A61P 3/10; A61K 31/155; A61K 31/715; A61K 31/45; A61K 31/13; A61K 31/165; A61K 31/427; A61K 31/426; A61K 31/4439

(52) U.S. Cl. 514/4; 514/3; 514/635; 514/54; 514/315; 514/579; 514/617; 514/331; 514/369; 514/342

(57) ABSTRACT

The invention provides a method of treatment of diabetes, particularly type II diabetes, or a diabetes related condition, comprising night time dosing of an inhibitor of glycogen phosphorylase, optionally in combination another anti-diabetic therapy.

### BACKGROUND OF THE INVENTION

The invention provides a method of treatment of diabetes, particularly type II diabetes, or a diabetes related condition, comprising night time dosing of an inhibitor of glycogen phosphorylase, optionally in combination with another anti-diabetic therapy.

Insulin dependent Type I diabetes and non-insulin dependent Type II diabetes continue to present treatment difficulties even though clinically accepted regimens that include diet, exercise, hypoglycemic agents, and insulin are available. Type II diabetes is by far the most common form of the disease and is found in over 90% of the diabetic patient population. Type II diabetic patient require chronic/long term treatment in order to maintain glycaemic control. The predominant pathophysiological defects in type II diabetes are insulin resistance and beta-cell dysfunction. The inexorable decline in beta-cell function which occurs in type II diabetes leads, in the majority of patients, to worsening of glycaemic control with time, requiring addition of more and more therapies as the disease progresses. Treatment is also patient dependent, therefore there is a continuing need for novel hypoglycemic agents, particularly ones that may be better tolerated with fewer adverse effects.

The liver and certain other organs produce glucose by breaking down glycogen (glycogenolysis) or by synthesizing glucose from small molecule precursors (gluconeogenesis), thereby raising the blood sugar levels. The inappropriate over-production of glucose by the liver as a result increased glycogenolysis is a contributor to hyperglycemia in type II diabetes. Glycogenolysis is catalyzed by glycogen phosphorylase enzyme. Accordingly, inhibiting glycogen phosphorylase (“GP”) may lower elevated blood sugar levels and represent a therapeutic option for the treatment of type II diabetes.

Similarly, hypertension and its associated pathologies such as, for example, atherosclerosis, lipidemia, hyperlipidemia and hypercholesterolemia have been associated with elevated insulin levels (hyperinsulinemia), which can lead to abnormal blood sugar levels. Furthermore, myocardial ischemia can result. Such maladies may be treated with hypoglycemic agents, including compounds that inhibit glycogen phosphorylase. The cardioprotective effects of glycogen phosphorylase inhibitors, for example following reperfusion injury, has also been described (see, for example, Ross et al., American Journal of Physiology. Heart and Circulatory Physiology, March 2004, 286(3), H1177-84). Accordingly, it is accepted that compounds that inhibit glycogen phosphorylase (see, for example, U.S. Pat. No. 6,297,269) are useful in the treatment of diabetes, hyperglycemia, hypercholesterolemia, hyperinsulinemia, hyperlipidemia, atherosclerosis or myocardial ischemia.

R. Kurukulasuriya, J. T. Link, et al., Current Medicinal Chem., 10:99-121(2003) describes “Prospects for Pharmacologic Inhibition of Hepatic Glucose Production.” R. Kurukulasuriya, J. T. Link, et al., Current Medicinal Chem., 10: 123-153(2003) describes “Potential Drug Targets and Progress Towards Pharmacologic Inhibition of Hepatic Glucose Production.”

U.S. Pat. No. 6,297,269 and European Patent Application No. EP 0832066 describe substituted N-(indole-2-carbonyl)amides and derivatives as glycogen phosphorylase inhibitors. U.S. Pat. Nos. 6,107,329 and 6,277,877 describe substituted N-(indole-2-carbonyl)glycinamides and derivatives as glycogen phosphorylase inhibitors. U.S. Pat. No. 6,399,601 describes bicyclic pyrrolyl amides as glycogen phosphorylase inhibitors. European Patent Application Nos. EP 0978276 and EP 1136071 describe inhibitors of human glycogen phosphorylase and their use. International Patent Publication No. WO 01/68055 describes glycogen phosphorylase inhibitors. U.S. Pat. No. 5,952,322 describes a method of reducing non-cardiac ischemial tissue damage using glycogen phosphorylase inhibitors.

U.S. Patent Publication No. 20030004162A1, European Patent Application No. EP 0846464, and International Publication No. WO 96/39384 describe glycogen phosphorylase inhibitors.

International Patent Application No. PCT/US2004/016243 (published after the priority date of the present invention) discloses pyrrolopyridine-2-carboxylic acid amide inhibitors of glycogen phosphorylase.

As described above GP inhibitors may represent a therapeutic option for the treatment of type II diabetes. In type II diabetes the contribution of hepatic glucose output to overall impaired glycaemia increases as the disease progresses, and it is therefore possible that GP inhibitors may have advantages over some current agents particularly in the late stage of the disease, prior to the use of insulin. However, chronic/long term GP inhibition may result in unwanted side effects or loss of glucose lowering effect with time (tachyphylaxis) as has been shown to be the case in clinical studies. The present invention provides a method to alleviate this potential problem by avoiding constant inhibition of glycogenolysis which may result in the liver's storage capacity for glycogen being exceeded, to the point that spillover of glucose release occurs. It is therefore desirable to find new treatment regimens for the administration of GP inhibitors. The present invention provides a dosing regimen which only results in GP inhibition for part of the 24 hour period.

### SUMMARY OF THE INVENTION

A method of treatment of diabetes, particularly type II diabetes, or a diabetes related condition, comprising night time dosing of an inhibitor of glycogen phosphorylase, optionally in combination with another anti-diabetic therapy.

### DETAILED DESCRIPTION OF THE INVENTION

The present invention provides a method of treatment of diabetes, particularly type II diabetes, or a diabetes related condition, comprising night time dosing of an inhibitor of glycogen phosphorylase.

The invention also provides a method for the treatment of diabetes particularly type II diabetes, or a diabetes related condition, in a mammal, preferably a human, comprising administering at night time a therapeutically effective amount of an inhibitor of glycogen phosphorylase to a mammal in need thereof.

The invention also provides the use of an inhibitor of glycogen phosphorylase in the manufacture of a medicament for the treatment of diabetes, particularly type II diabetes, or a diabetes related condition, wherein the administration pattern comprises administration of the inhibitor of glycogen phosphorylase at night time.

The invention also provides the use of an inhibitor of glycogen phosphorylase for the treatment of diabetes, particularly type II diabetes, or a diabetes related condition, wherein the administration pattern comprises administration of the inhibitor of glycogen phosphorylase at night time.

Night time dosing of the glycogen phosphorylase inhibitor preferably comprises administration prior to bedtime e.g. at bedtime, and particularly after any other anti-diabetic therapy has been administered. The glycogen phosphorylase inhibitor is preferably administered after the subject has consumed their last meal of the day such that inhibition of glycogen phosphorylase occurs during the fasting period. The glycogen phosphorylase inhibitor is preferably administered only once during a 24 hour period.

The method of the invention is preferably for the treatment of type II diabetes.

The method according to the invention provides a novel and advantageous method for the treatment of type II diabetes which results in an effective process for the control of basal blood glucose levels whilst avoiding the potential for unwanted side effects e.g. those related to hypoglycaemia. The use of a GP inhibitor in this manner may also avoid the development of compensatory mechanisms such as an increase in gluconeogenesis which could occur in response to sustained inhibition of glycogenolysis. It also provides advantages over the use of other conventional agents, such as sulfonylureas, other insulin secretagogues and insulins, which have safety aspects regarding night time dosing since they might lead to hypoglycaemia overnight which could be fatal for the patient.

The night time dosing of the inhibitor of glycogen phosphorylase may be used as polypharmacy together with another anti-diabetic therapy.

The present invention provides a method of treatment of diabetes, particularly type II diabetes, or a diabetes related condition, comprising night time dosing of an inhibitor of glycogen phosphorylase and administration of another anti-diabetic therapy.

The invention also provides a method for the treatment of diabetes particularly type II diabetes, or a diabetes related condition, in a mammal, preferably a human, comprising administering at night time a therapeutically effective amount of an inhibitor of glycogen phosphorylase, and administering another anti-diabetic therapy, to a mammal in need thereof.

The invention also provides the use of an inhibitor of glycogen phosphorylase in the manufacture of a medicament for the treatment of diabetes, particularly type II diabetes, or a diabetes related condition, wherein the administration pattern comprises administration of the inhibitor of glycogen phosphorylase at night time and administration of another anti-diabetic therapy.

The invention also provides the use of an inhibitor of glycogen phosphorylase for the treatment of diabetes, particularly type II diabetes, or a diabetes related condition, wherein the administration pattern comprises administration of the inhibitor of glycogen phosphorylase at night time and administration of another anti-diabetic therapy.

The inhibitor of glycogen phosphorylase is preferably administered in combination with another e.g. day time, such as meal related, anti-diabetic therapy.

The present invention provides a method of treatment of diabetes, particularly type II diabetes, or a diabetes related condition, comprising night time dosing of an inhibitor of glycogen phosphorylase and administration of another anti-diabetic therapy in the day time.

The invention also provides a method for the treatment of diabetes particularly type II diabetes, or a diabetes related condition, in a mammal, preferably a human, comprising administering at night time a therapeutically effective amount of an inhibitor of glycogen phosphorylase, and administering in the day time another anti-diabetic therapy, to a mammal in need thereof.

The invention also provides the use of an inhibitor of glycogen phosphorylase in the manufacture of a medicament for the treatment of diabetes, particularly type II diabetes, or a diabetes related condition, wherein the administration pattern comprises administration of the inhibitor of glycogen phosphorylase at night time and administration of another anti-diabetic therapy in the day time.

The invention also provides the use of an inhibitor of glycogen phosphorylase for the treatment of diabetes, particularly type II diabetes, or a diabetes related condition, wherein the administration pattern comprises administration of the inhibitor of glycogen phosphorylase at night time and administration of another anti-diabetic therapy in the day time.

Administration of the other anti-diabetic therapy in the day time refers to administration during the waking hours of the subject, such administration may be once, twice or three times a day and may be meal related or prandial, i.e. taken at the time of one or more meals in the day.

When used in combination with additional anti-diabetic therapy the method of the invention may also allow the dose of the additional anti-diabetic therapy to be reduced compared to that required in the absence of the administration of a glycogen phosphorylase inhibitor.

When the method of the invention is used in conjunction with the administration of another anti-diabetic therapy the additional anti-diabetic agent is preferably an agent traditionally used for day time e.g. meal related or prandial treatment, the agent may be selected from PPAR agonists, biguanides, sulfonylureas and other insulin secretagogues, insulin sensitisers, alpha-glucosidase inhibitors, dipeptidyl peptidase IV inhibitors, glucokinase activators, GLP-1 and GLP-1 mimetics/analogues, insulin and insulin analogues.

Suitably, the other antidiabetic agent comprises one or more, generally one or two of the agents listed above.

A suitable alpha-glucosidase inhibitor is acarbose.

Other suitable alpha-glucosidase inhibitors are emiglitate and miglitol. A further suitable alpha-glucosidase inhibitor is voglibose.

Suitable biguanides include metformin, buformin and phenformin, especially metformin.

Suitable insulin secretagogues include sulphonylureas.

Suitable sulphonylureas include glibenclamide, glipizide, gliclazide, glimepiride, tolazamide and tolbutamide. Further sulphonylureas include acetohexamide, carbutamide, chlorpropamide, glibornuride, gliquidone, glisentide, glisolamide, glisoxepide, glyclopyamide and glycylamide. Also included is the sulphonylurea glipentide.

A further suitable insulin secretagogue is repaglinide. An additional insulin secretagogue is nateglinide.

Insulin sensitisers include PPARy agonist insulin sensitisers including the compounds disclosed in WO 97/31907 and especially 2-(1-carboxy-2-{4-{2-(5-methyl-2-phenyl-oxazol-4-yl)ethoxy]phenylethylamino)benzoic acid methyl ester and 2 (S)-(2-benzoylphenylamino)-3-{4-[2-(5-methyl-2-phenyl-oxazol-4-yl)ethoxy]phenyl}propionic acid.

Insulin sensitisers also include thiazolidinedione insulin sensitisers.

Other suitable thiazolidinedione insulin sensitisers include (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone).

Particular thiazolidinedione insulin sensitisers are 5-[4-[2-(5-ethylpyridin-2-yl)ethoxy]benzyl]thiazolidine-2,4-dione (or pioglitazone) and (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone).

A preferred thiazolidinedione insulin sensitiser is 5-[4-[2-(N-methyl-N-(2-pyridyl)amino)ethoxy]benzyl]thiazolidine-2,4-dione (or rosiglitazone) and salts thereof.

GLP-1 mimetics and analogues include NN-2211 (liraglutide), exendin-4 and exendin-4 mimetics, e.g. exenatide.

Other antidiabetic agents which may be mentioned are α2 agonists, fatty acid oxidation inhibitors, α-glucosidase inhibitors, β-agonists, phosphodiesterase inhibitors, lipid lowering agents, antiobesity agents, amylin antagonists, lipoxygenase inhibitors, somostatin analogs, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, CRF antagonists and CRF binding proteins.

The glycogen phosphorylase inhibitor is preferably as described in International Patent Application No. PCT/US2004/016243, i.e. a compound of Formula (I):

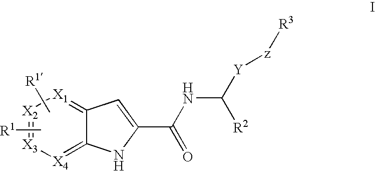

or a stereoisomer, or a pharmaceutically acceptable salt thereof, wherein:

one of X₁, X₂, X₃ and X₄ must be N and the others must be C;

R¹ and R^{1′} are each independently, halogen, hydroxy, cyano, C₀₋₄alkyl, C₁₋₄alkoxy, fluoromethyl, difluoromethyl, trifluoromethyl, ethenyl, or ethynyl;

R² is C₀₋₄alkyl, COOR⁶, COR⁶, C₁₋₄alkoxyC₁₋₄alkyl-, hydroxyC₁₋₄alkyl-, cycloalkylC₀₋₄alkyl-, arylC₀₋₄alkyl-, hetarylC₀₋₄alkyl-, wherein any of the aryl or hetaryl rings are optionally substituted with 1-2 independent halogen, cyano, C₁₋₄alkyl, C₁₋₄alkoxy, —N(C₀₋₄alkyl)(C₀₋₄alkyl), —SO₂C₁₋₄alkyl, —SO₂N(C₀₋₄alkyl)(C₀₋₄alkyl), hydroxy, fluoromethyl, difluoromethyl, or trifluoromethyl substituents;

Y is C₀₋₂alkyl or —CH(OH)—;

Z is CH₂, —C(O)—, —O—, >N(C₀₋₄alkyl), >N(C₃₋₆cycloalkyl), or absent; but when Y is —CH(OH)—, Z or R³ must be bonded to Y through a carbon-carbon bond;

R³ is hydrogen, —COOC₀₋₄alkyl, C₁₋₄alkoxy, C₁₋₄alkyl, arylC₁₋₄alkylthio-, —C₀₋₄alkylaryl, —C₀₋₄alkylhetaryl, —C₀₋₄alkylcycloalkyl or —C₀₋₄alkylheterocyclyl, wherein any of the rings is optionally substituted with 1-3 independent halogen, cyano, C₁₋₄alkyl, fluoromethyl, difluoromethyl, trifluoromethyl, —C₀₋₄alkylNHC(O)O(C₁₋₄alkyl), —C₀₋₄alkylNR⁷R⁸, —C(O)R⁹, C₁₋₄alkoxyC₀₋₄alkyl-, —COOC₀₋₄alkyl, —C₀₋₄alkylNHC(O)R⁹, —C₀₋₄alkylC(O)N(R¹⁰)₂, —C₁₋₄alkoxyC₁₋₄alkoxy, hydroxyC₀₋₄alkyl-, —NHSO₂R¹⁰, —SO₂(C₁₋₄alkyl), —SO₂NR¹¹R¹², 5- to 6-membered heterocyclyl, phenylC₀₋₂alkoxy, or phenylC₀₋₂alkyl substituents, wherein phenyl is optionally substituted with 1-2 independent halogen, cyano, C₁₋₄alkyl, C₁₋₄alkoxy, —N(C₀₋₄alkyl)(C₀₋₄alkyl), —SO₂C₁₋₄alkyl, —SO₂N(C₀₋₄alkyl)(C₀₋₄alkyl), hydroxy, fluoromethyl, difluoromethyl or trifluoromethyl substituents, or two bonds on a ring carbon of the heterocyclyl group optionally can form an oxo (=O) substituent;

or R³ is —NR⁴(—C₀₋₄alkylR⁵);

R⁴ is C₀₋₃alkyl, —C₂₋₃alkyl-NR⁷R⁸, C₃₋₆cycloalkyl optionally substituted by hydroxyC₀₋₄alkyl- further optionally substituted by hydroxy, C₁₋₂alkoxyC₂₋₄alkyl-, or C₁₋₂alkyl-S(O)ₙ—C₂₋₃alkyl-;

n is 0, 1, or 2;

R⁵ is hydrogen, hydroxyC₂₋₃alkyl-, C₁₋₂alkoxyC₀₋₄alkyl-, or aryl, hetaryl, or heterocyclyl;

wherein a heterocyclic nitrogen-containing R⁵ ring optionally is mono-substituted on the ring nitrogen with C₁₋₄alkyl, benzyl, benzoyl, C₁₋₄alkyl-C(O)—,

—SO₂C₁₋₄alkyl, —SO₂N(C₀₋₄alkyl)(C₀₋₄alkyl), C₁₋₄alkoxycarbonyl or aryl(C₁₋₄alkoxy)carbonyl; and wherein the R⁵ rings are optionally mono-substituted on a ring carbon with halogen, cyano, C₁₋₄alkyl-C(O)—, C₁₋₄alkyl-SO₂—, C₁₋₄alkyl, C₁₋₄alkoxy, hydroxy, —N(C₀₋₄alkyl)(C₀₋₄alkyl), hydroxyC₀₋₄alkyl-, or C₀₋₄alkylcarbamoyl-, provided that no quaternised nitrogen is included; or two bonds on a ring carbon of the heterocyclyl group optionally can form an oxo (=O) substituent;

R⁶ is C₁₋₄alkyl, aryl, or hetaryl;

R⁷ and R⁸ are independently C₀₋₄alkyl, C₃₋₆cycloalkyl, or CO(C₁₋₄alkyl);

R⁹ is C₁₋₄alkyl, or C₃₋₆cycloalkyl;

R¹⁰ is C₀₋₄alkyl, or C₃₋₆cycloalkyl;

R¹¹ and R¹² are independently C₀₋₄alkyl or together with the nitrogen to which they are attached may form a 4- to 6-membered heterocycle; and

wherein there are no nitrogen-oxygen, nitrogen-nitrogen or nitrogen-halogen bonds in linking the three components —Y—Z—R³ to each other.

The molecular weight of the compounds of Formula (I) is preferably less than 800, more preferably less than 600.

In the compounds of Formula (I):

Preferably X₃ is N.

Preferably R¹ and R^{1′} are each independently, halogen, cyano, hydrogen, methyl, methoxy, or ethynyl. More preferably R¹ and R^{1′} are each independently, halogen, cyano, or hydrogen.

Preferably at least one of R¹ and R^{1′} is hydrogen. More preferably one of R¹ and R^{1′} is hydrogen.

A preferred group of compounds are those where X₃ is N, one of R¹ and R^{1′} is hydrogen and the other is a 5-halo or 5-cyano group.

Preferably Y is C₀₋₂alkyl, more preferably Y is a direct bond.

Preferably Z is —(O)—.

A preferred group of compounds are those wherein

X₃ is N;

Y is C₀₋₂alkyl; and

Z is —C(O)—.

Preferably R² is C₀₋₄alkyl or arylC₀₋₄alkyl-, wherein the aryl ring is optionally substituted with 1-2 independent halogen, cyano, C₁₋₄alkyl, C₁₋₄alkoxy, —N(C₀₋₄alkyl)(C₀₋₄alkyl), —SO₂C₁₋₄alkyl, —SO₂N(C₀₋₄alkyl)(C₀₋₄alkyl), hydroxy, fluoromethyl, difluoromethyl, or trifluoromethyl substituents. More preferably R² is benzyl optionally substituted with 1-2 halogen substituents. A particular R² substituent which may be mentioned is —(S)-(4-fluorobenzyl).

Preferably R³ is —C₀₋₄alkylheterocyclyl optionally substituted with 1-3 independent halogen, cyano, C₁₋₄alkyl, fluoromethyl, difluoromethyl, trifluoromethyl, —C₀₋₄alkylNHC(O)O(C₁₋₄alkyl), —C₀₋₄alkylNR⁷R⁸, —C(O)R⁹, C₁₋₄alkoxyC₀₋₄alkyl-,

—COOCO₀₋₄alkyl, —C₀₋₄alkylNHC(O)R⁹, —C₀₋₄akylC(O)N(R¹⁰)₂, —C₁₋₄alkoxyC₁₋₄alkoxy, hydroxyC₀₋₄alkyl-, —NHSO₂R¹⁰, —SO₂(C₁₋₄alkyl), —SO₂NR¹¹R¹², 5- to 6-membered heterocyclyl, phenylC₀₋₂alkoxy, or phenylC₀₋₂alkyl substituents, wherein phenyl is optionally substituted with 1-2 independent halogen, cyano, C₁₋₄alkyl, C₁₋₄alkoxy, —N(C₀₋₄alkyl)(C₀₋₄alkyl), —SO₂C₁₋₄alkyl, —SO₂N(C₀₋₄alkyl)(C₀₋₄alkyl), hydroxy, fluoromethyl, difluoromethyl, or trifluoromethyl substituents, or two bonds on a ring carbon of the heterocyclyl group optionally can form an oxo (=O) substituent; or R³ is —NR⁴(—C₀₋₄alkylR⁵).

More preferably R³ is a nitrogen containing heterocyclyl group, especially a 4-8-membered nitrogen containing heterocyclyl group, linked to Z via a ring nitrogen atom, optionally substituted with 1-3 independent halogen, cyano, C₁₋₄alkyl, fluoromethyl, difluoromethyl, trifluoromethyl, —C₀₋₄alkylNHC(O)O(C₁₋₄alkyl), —C₀₋₄alkylNR⁷R⁸, —C(O)R⁹, C₁₋₄alkoxyC₀₋₄alkyl-, —COOC₀₋₄alkyl, —C₀₋₄alkylNHC(O)R⁹, —C₀₋₄alkylC(O)N(R¹⁰)₂, —C₁₋₄alkoxyC₁₋₄alkoxy, hydroxyC₀₋₄alkyl-, —NHSO₂R¹⁰, —SO₂(C₁₋₄alkyl), —SO₂NR¹¹R¹², 5- to 6-membered heterocyclyl, phenylC₀₋₂alkoxy, or phenylC₀₋₂alkyl substituents, wherein phenyl is optionally substituted with 1-2 independent halogen, cyano, C₁₋₄alkyl, C₁₋₄alkoxy, —N(C₀₋₄alkyl)(C₀₋₄alkyl), —SO₂C₁₋₄alkyl, —SO₂N(C₀₋₄alkyl)(C₀₋₄alkyl), hydroxy, fluoromethyl, difluoromethyl, or trifluoromethyl substituents, or two bonds on a ring carbon of the heterocyclyl group optionally can form an oxo (=O ) substituent; or R³ is —NR⁴(—C₀₋₄alkylR⁵).

Examples of nitrogen containing heterocyclyl groups which R³ may represent include azetidin-1-yl, pyrrolidin-1-yl, piperidin-1-yl, 1,4-diazapan-1-yl, piperazin-1-yl, morpholin-4-yl, thiomorpholin-4-yl, 1,1-dioxo-thiomorpholin-4-yl, or thiazolidin-3-yl; which groups may be optionally substituted as described above Preferred substituent groups for R³ include —C₁₋₄alkoxy, hydroxy and oxo.

Even more preferably R³ is pyrrolidin-1-yl or piperidin-1-yl optionally substituted with hydroxyl, e.g. 4-hydroxypiperidin-1-yl and 3-(S)-hydroxypyrrolidin-1-yl.

A particularly preferred glycogen phosphorylase inhibitor for use in the invention is 5-chloro-1H-pyrrolo[2,3-c]pyridine-2-carboxylic acid [1(S)-(4-fluorobenzyl)-2-(4-hydroxypiperidin-1-yl)-2-oxoethyl]amide, or a pharmaceutically acceptable salt thereof especially the hydrochloride salt.

While the preferred groups for each variable have generally been listed above separately for each variable, preferred compounds of this invention include those in which several or each variable in Formula (I) is selected from the preferred, more preferred, most preferred, especially or particularly listed groups for each variable. Therefore, this invention is intended to include all combinations of preferred, more preferred, most preferred, especially and particularly listed groups.

As used herein, unless stated otherwise, “alkyl” as well as other groups having the prefix “alk” such as, for example, alkoxy, alkanyl, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

As used herein, for example, “C₀₋₄alkyl” is used to mean an alkyl having 0-4 carbons—that is, 0, 1, 2, 3, or 4 carbons in a straight or branched configuration. An alkyl having no carbon is hydrogen when the alkyl is a terminal group. An alkyl having no carbon is a direct bond when the alkyl is a bridging (connecting) group.

The terms “cycloalkyl” and “carbocyclic ring” mean carbocycles containing no heteroatoms, and include mono-, bi-, and tricyclic saturated carbocycles, as well as fused and bridged systems. Such fused ring systems can include one ring that is partially or fully unsaturated, such as a benzene ring, to form fused ring systems, such as benzofused carbocycles. Cycloalkyl includes such fused ring systems as spirofused ring systems. Examples of cycloalkyl and carbocyclic rings include C₃₋₁₀cycloalkyl groups, particularly C₃₋₈cycloalkyl groups, such as cyclopropyl, cyclobutyl, cyclopentyl, cyclohexyl, and decahydronaphthalene, adamantane, indanyl, 1,2,3,4-tetrahydronaphthalene and the like.

The term “halogen” includes fluorine, chlorine, bromine, and iodine atoms.

The term “carbamoyl” unless specifically described otherwise means —C(O)—NH— or —NH—C(O)—.

The term “aryl” is well known to chemists. The preferred aryl groups are phenyl and naphthyl, more preferably phenyl.

The term “hetaryl” is well known to chemists. The term includes 5- or 6-membered heteroaryl rings containing 1-4 heteroatoms chosen from oxygen, sulfur, and nitrogen in which oxygen and sulfur are not next to each other. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The term “hetaryl” includes hetaryl rings with fused carbocyclic ring systems that are partially or fully unsaturated, such as a benzene ring, to form a benzofused hetaryl. For example, benzimidazole, benzoxazole, benzothiazole, benzofuran, quinoline, isoquinoline, quinoxaline, and the like.

Unless otherwise stated, the terms “heterocyclic ring”, “heterocyclyl” and “heterocycle” are equivalent, and include 4-10-membered, e.g. 4-8-membered, saturated or partially saturated rings containing one or two heteroatoms chosen from oxygen, sulfur, and nitrogen. The sulfur and oxygen heteroatoms are not directly attached to one another. Any nitrogen heteroatoms in the ring may optionally be substituted with C₁₋₄alkyl. Examples of heterocyclic rings include azetidine, oxetane, tetrahydrofuran, tetrahydropyran, oxepane, oxocane, thietane, thiazolidine, oxazolidine, oxazetidine, pyrazolidine, isoxazolidine, isothiazolidine, tetrahydrothiophene, tetrahydrothiopyran, thiepane, thiocane, azetidine, pyrrolidine, piperidine, N-methylpiperidine, azepane, 1,4-diazapane, azocane, [1,3]dioxane, oxazolidine, piperazine, homopiperazine, morpholine, thiomorpholine, 1,2,3,6-tetrahydropyridine and the like. Other examples of heterocyclic rings include the oxidized forms of the sulfur-containing rings. Thus, tetrahydrothiophene-1-oxide, tetrahydrothiophene-1,1-dioxide, thiomorpholine-1-oxide, thiomorpholine-1,1-dioxide, tetrahydrothiopyran-1-oxide, tetrahydrothiopyran-1,1-dioxide, thiazolidine-1-oxide, and thiazolidine-1,1-dioxide are also considered to be heterocyclic rings. The term “heterocyclic” also includes fused ring systems and can include a carbocyclic ring that is partially or fully unsaturated, such as a benzene ring, to form benzofused heterocycles. For example, 3,4-dihydro-1,4-benzodioxine, tetrahydroquinoline, tetrahydroisoquinoline and the like.

Compounds of Formula (I) may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula (I) exists, the present invention includes any possible tautomers and pharmaceutically acceptable salts thereof, and mixtures thereof, except where specifically drawn or stated otherwise.

When the compound of Formula (1) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of Formula (I) is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of Formula (I) is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Other glycogen phosphorylase inhibitors are known which may be used according to the method of the invention, these include compounds described in U.S. Pat. No. 6,297,269, EP 0832066, U.S. Pat. No. 6,107,329, U.S. Pat. No. 6,277,877, U.S. Pat. No. 6,399,601, EP 0978276, EP 1136071, US 20030004162A1, US 2003/0187051, US 2004/0002495A1, US 2004/0142938A1, EP 0846464, WO 96/39384, WO 96/39385, WO97/09040, WO 00/27206, WO 01/68055, WO 01/68092, WO 02/20530, WO 03/037864, WO03/072570, WO 03/074484, WO 03/074485, WO 03/074513, WO 03/074517, WO 03/074531, WO 03/074532, WO 03/091213, WO 05/013975, WO05/013981, WO 05/018637, WO 05/019172, WO 05/020985, WO 05/020986, WO 05/020987, WO 05/067932, WO 05/073229, WO 05/073230, WO 05/073231, WO 05/085194 and WO 05/085245 the disclosures of which are hereby incorporated by reference.

The glycogen phosphorylase inhibitor for use in accordance with the invention preferably has a duration of action which is less than 12 hours e.g. less than 10 hours. The duration of action of the inhibitor may be measured by methods known to those skilled in the art.

The glycogen phosphorylase inhibitors for use in the invention will be administered as a pharmaceutical composition that is comprised of the glycogen phosphorylase inhibitor in combination with a pharmaceutically acceptable carrier.

Preferably the composition is comprised of a pharmaceutically acceptable carrier and a non-toxic therapeutically effective amount of a glycogen phosphorylase inhibitor.

The compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions are preferably suitable for oral administration. The pharmaceutical compositions may be conveniently presented in unit dosage form and prepared by any of the methods well known in the art of pharmacy.

The glycogen phosphorylase inhibitors can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions can be presented as discrete units suitable for oral administration such as capsules, sachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. The compositions may be prepared by any of the methods of pharmacy. In general, such methods include a step of bringing into association the glycogen phosphorylase inhibitor with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The glycogen phosphorylase inhibitors can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

Preferably the pharmaceutical composition comprising the glycogen phosphorylase inhibitor is presented as a discrete unit suitable for oral administration, preferably as a solid dosage form, e.g. in the form of a tablet, cachet or capsule.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each sachet or capsule preferably contains from about 0.05 mg to about 5 g of the glycogen phosphorylase inhibitor.

For example, a formulation intended for oral administration to humans may contain from about 0.5 mg to about 5 g of active agent, compounded with an appropriate and convenient amount of carrier material, which may vary from about 5 to about 95% of the total composition. Unit dosage forms will generally contain from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a glycogen phosphorylase inhibitor may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, diabetes and hyperglycemia may be effectively treated by the administration of from about 0.01 to 50 mg of the compound per kilogram of body weight per day, or alternatively about 0.5 mg to about 3.5 g per patient per day. Similarly, hypercholesterolemia, hyperinsulinemia, hyperlipidemia, hypertension, atherosclerosis or tissue ischemia e.g. myocardial ischemia may be effectively treated by the administration of from about 0.01 to 50 mg of the compound per kilogram of body weight per day, or alternatively about 0.5 mg to about 3.5 g per patient per day, e.g. 50 mg to 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

Diseases or conditions which may be treated according to the method of the invention include diabetes (including Type I and Type II impaired glucose tolerance, insulin resistance and diabetic complications such as neuropathy, nephropathy, retinopathy and cataracts), hyperglycemia, hypercholesterolemia, hyperinsulinemia and hyperlipidemia.

In the methods of the invention the term “treatment” includes both therapeutic and prophylactic treatment.

The glycogen phosphorylase inhibitors may be administered alone or in combination with one or more other therapeutically active compounds. The therapeutically active compounds may be administered simultaneously, sequentially or separately, preferably they are administered separately.

As described above the GP inhibitors may be administered as polypharmacy with other active compounds for the treatment of diabetes, for example PPAR agonists, biguanides, sulfonylureas and other insulin secretagogues, insulin sensitisers, alpha-glucosidase inhibitors, dipeptidyl peptidase IV inhibitors, glucokinase activators, GLP-1 and GLP-1 analogues, insulin, insulin analogues, α2 agonists, fatty acid oxidation inhibitors, α-glucosidase inhibitors, β-agonists, phosphodiesterase inhibitors, lipid lowering agents, antiobesity agents, amylin antagonists, lipoxygenase inhibitors, somostatin analogs, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, CRF antagonists and CRF binding proteins. The GP inhibitors may also be administered in combination with thyromimetic compounds, aldose reductase inhibitors, glucocorticoid receptor antagonists, NHE-1 inhibitors or sorbitol dehydrogenase inhibitors. These additional agents may be formulated and administered by methods known to those skilled in the art.

All publications, including, but not limited to, patents and patent application cited in this specification, are herein incorporated by reference as if each individual publication were specifically and individually indicated to be incorporated by reference herein as fully set forth.

The compounds of Formula (I) can be prepared as outlined in Scheme 1 below wherein R¹, R^{1′}, R², R³, X₁, X₂, X₃, X₄, Y and Z are as defined above for Formula (I):

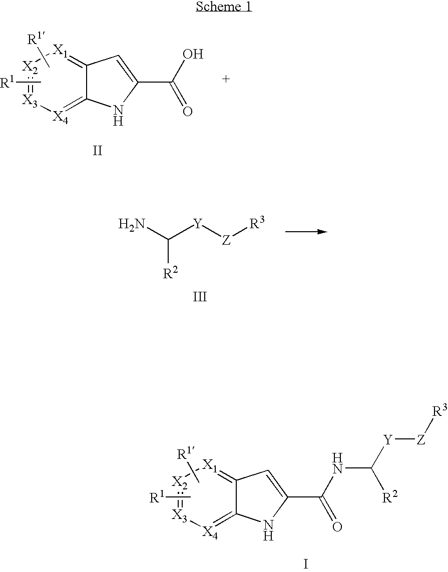

According to Scheme 1, the compounds of Formula (I) may be prepared by coupling the appropriate pyrrolopyridine-2-carboxylic acid of Formula (II), or a protected or activated derivative thereof, with the appropriate amine of Formula (III). Compounds of Formula (II) can be obtained by the syntheses described in Schemes 3 and 5 below. Compounds of Formula (III) are generally commercially available or can be obtained by the syntheses described in Schemes 8 and 9 below.

Typically, the compound of Formula (II), or a protected or activated derivative thereof, is combined with a compound of Formula (III) in the presence of a suitable coupling agent. Examples of suitable coupling reagents are 1-(3-dimethylaminopropyl)-3-ethylcarbodiimide hydrochloride/hydroxybenzotriazole (EDCI/HOBt), 1,1-carbonyldiimidazole (CDI), dicyclohexylcarbodiimide/hydroxybenzotriazole (DCC/HOBt), O-(1H-benzotriazol-1-yl)-N,N,N′,N′-tetramethyluronium tetrafluoroborate (R. Knorr et al., Tetrahedron Lett., 1989, 30, 1927-1930) and polymer supported carbodiimide-1-hydroxybenzotriazole (for representative procedures, see for example, Argonaut Technical Note 501 available from Argonaut Technologies, Inc., Foster City, Calif.). The couplings are performed in an inert solvent, preferably an aprotic solvent at a temperature of about 0° C. to about 45° C. for about 1 to 72 h in the presence of a tertiary amine base such as diisopropylethylamine (DIPEA) or triethylamine. Exemplary solvents include acetonitrile, chloroform, dichloromethane, N,N-dimethylformamide (DMF) or mixtures thereof. Use of these coupling agents and appropriate selection of solvents and temperatures are known to those skilled in the art or can be readily determined from the literature. These and other exemplary conditions useful for coupling carboxylic acids are described in Houben-Weyl, Vol XV, part II, E. Wunsch, Ed., G. Thieme Verlag, 1974, Stuttgart, and M. Bodansky, Principles of Peptide Synthesis, Springer-Verlag, Berlin, 1984 and The Peptides, Analysis, Synthesis and Biology (Ed., E. Gross and J. Meienhofer), Vols 1-5, Academic Press NY 1979-1983.

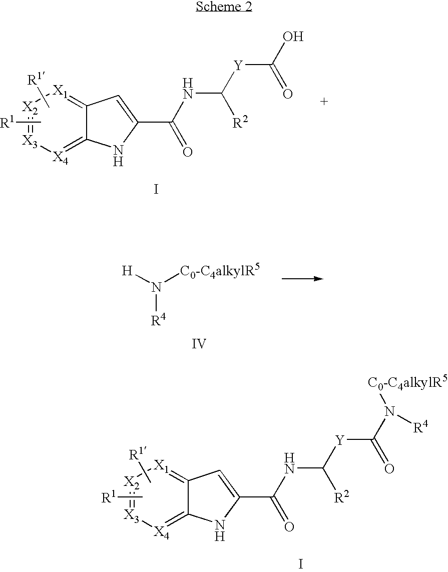

In a second process, the compounds of Formula (I) (wherein Z is C═O and R³ is —NR⁴(—C₀₋₄alkylR⁵)) may be prepared according to Scheme 2 by coupling the appropriate carboxylic acid of Formula (I), or a protected or activated derivative thereof, (wherein Z is absent and R³ is —CO₂H) with the appropriate amine of Formula (IV). Examples of suitable coupling agents and conditions are as described above. Compounds of Formula (IV) are commercially available or are readily prepared by known techniques.

Compounds of Formula (II) can be prepared as illustrated in Scheme 3.

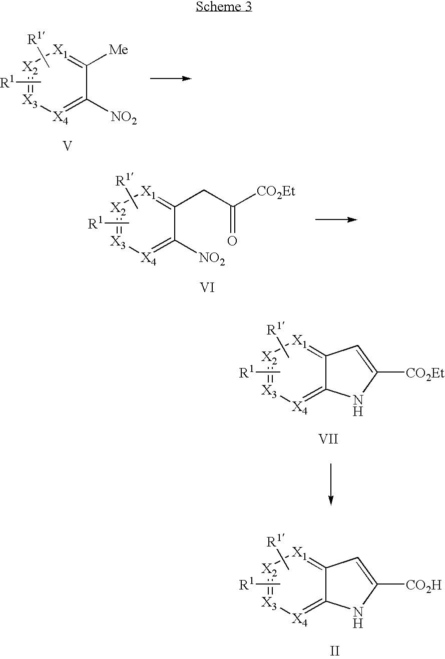

Compounds of Formula (VI) may be prepared by condensation of ortho methyl nitro compounds of Formula (V) with an oxalate ester in a solvent such as diethyl ether in the presence of a base such as potassium ethoxide or DBU. Compounds of Formula (VII) are prepared from compounds of Formula (VI) under reducing conditions, such as iron powder and ammonium chloride, or by hydrogenation in ethanol using palladium catalysis. Compounds of Formula (VII) undergo ester hydrolysis using aqueous alkali to give pyrrolopyridine-2-carboxylic acids of Formula (II). Further information on the conversion of compounds of Formula (V) to compounds of Formula (II) are described in the literature (Kermack, et al., J. Chem, Soc., 1921, 119, 1602; Cannon et al., J. Med. Chem., 1981, 24, 238; Julian et al., in Heterocyclic Compounds, Vol 3 (Wiley, New York, N.Y., 1962, R. C. Elderfield, Ed.) p 18.

Alternatively, the compound of Formula (VII) wherein X₂ is nitrogen can be prepared as illustrated in Scheme 4.

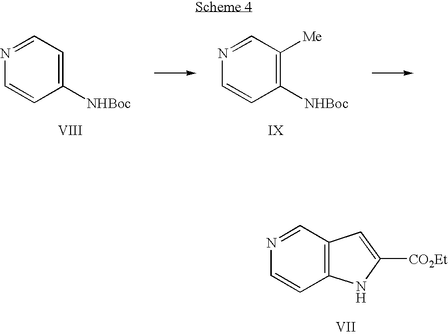

Deprotonation of compounds of Formula (VIII) with an organolithium such as n-butyllithium in a suitable solvent such as THF, followed by quenching with methyl iodide gives compounds of Formula (IX). Such compounds can undergo further deprotonation with tert-butyllithium, in a suitable solvent such as THF, followed by quenching with diethyl oxalate and subsequent heating of the intermediate under reflux in hydrochloric acid, to give compounds of Formula (VII).

Compounds of Formula (II) may also be prepared according to Scheme 5 by Heck coupling of an ortho-iodo aminopyridine (XIV) followed by cyclisation at a temperature of between 100 to 150° C. in the presence of catalyst such as palladium acetate and a base such as DABCO in a solvent such as DMF (See Chen et al, J. Org. Chem. 1997, 62, 2676). The ortho-iodo aminopyridines (XIV) can be made by direct iodination of the appropriate aminopyridine (XIII) using iodine in the presence of silver sulfate in a solvent such as ethanol at ambient temperature (see Sy, W., Synth. Commun., 1992, 22, 3215).

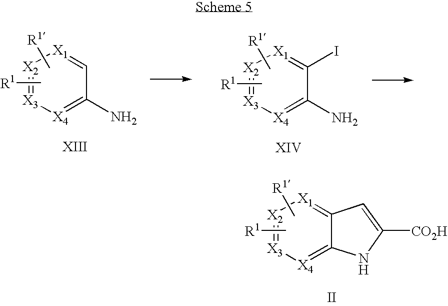

Alternatively compounds of Formula (XIV) may be prepared according to Scheme 6 by deprotection of N-pivaloyl compounds (XV) by heating under reflux using hydrochloric acid. The N-pivaloyl compounds (XV) are in turn made by deprotonation of compounds of Formula (XVI) with an organolithiumn such as tert-butyllithium in a suitable solvent such as THF, followed by quenching with iodine at a low temperature. Compounds of formula (XVI) may be made by protection of commercially available aminopyridines (XII) with trimethylacetyl chloride and a base such as triethylamine in a solvent such as dichloromethane.

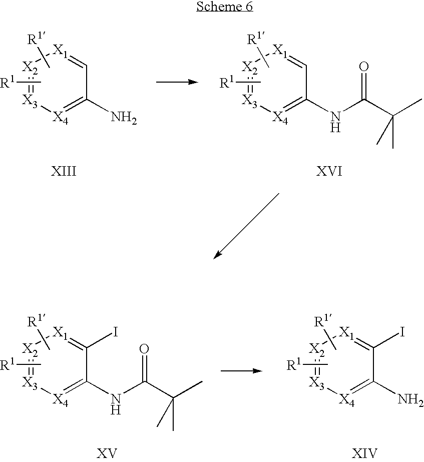

Alternatively compounds of Formula (XIV) may be prepared according to Scheme 7 by deprotection of N-BOC protected compounds (XVII) using an acid such as trifluoroacetic acid in a solvent such as dichloromethane at ambient temperature. The N-BOC compounds (XVII) are in turn made by deprotonation of compounds of Formula (XVII) with an organolithium such as n-butyllithium in the presence of N,N,N′,N′-tetramethylethylenediamine (TMEDA) in a suitable solvent such as ether at temperatures around −70° C. followed by the addition of iodine at temperatures around −10° C. The N-BOC aminopyridines (XVIII) are routinely made from the commercially available aminopyridines (XIII) using di-tert-butyldicarbonate by heating in a solvent such as 1,4-dioxane.

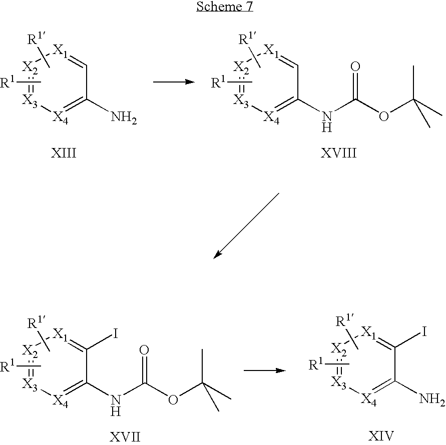

Protected or activated derivatives of the compounds of Formula (II) may be prepared by methods known to those skilled in the art.

Compounds of Formula (III) can be prepared as illustrated in Scheme 8.

Compounds of Formula (X) are generally commercially available or are readily prepared by known techniques. PG represents a protecting group such as, for example, tert-butyloxycarbonyl (Boc). Compounds of Formula (XI) are made from carboxylic acids of Formula (X) using standard coupling conditions as described above for Scheme 1.

Compounds of Formula (III) can be prepared as illustrated in Scheme 8.

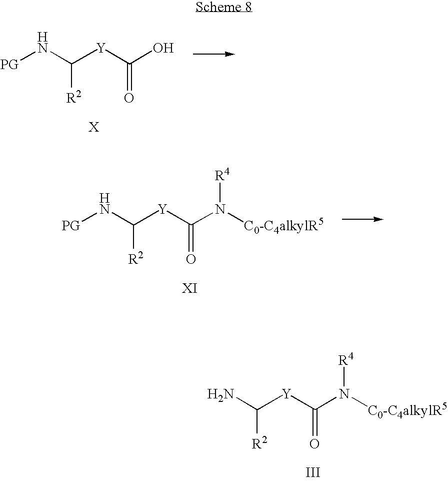

Compounds of Formula (III) may be prepared from compounds of Formula (XI) by removal of the protecting group, where PG=Boc, under acidic conditions using for example trifluoroacetic acid in dichloromethane at temperatures of around 25° C.

Compounds of Formula (III) wherein R² is H, Y is C₀ alkyl, Z is —C(O)— and R³ is —C₀alkylaryl or —C₀alkylhetaryl can be prepared according to Scheme 9.

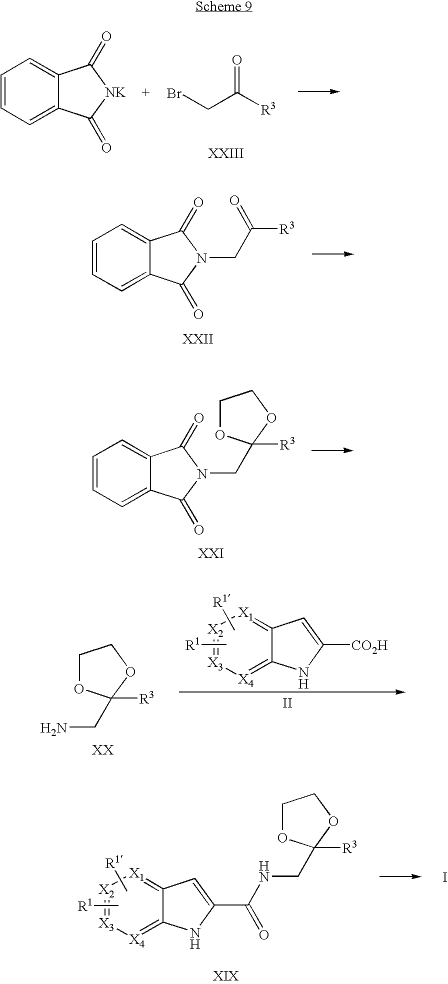

Compounds of Formula (XXIII) are reacted with potassium phthalimide in a solvent such as DMF to give compounds of Formula (XXII) which can then be reacted with ethylene glycol in the presence of a catalytic amount of acid such as p-toluene sulfonic acid in a solvent such as toluene whilst removing water to give compounds of Formula (XXI). The phthalimide protecting group can then be removed using hydrazine hydrate by heating as a neat solution or by heating in a solvent such as ethanol to give compounds of Formula (XX). These amines are then coupled with compounds of Formula (II) under standard coupling conditions as described in Scheme 1, and then the ketal group is removed in the presence of acid such as hydrochloric acid in a solvent such as acetone at reflux temperature to give the compounds of Formula (I).

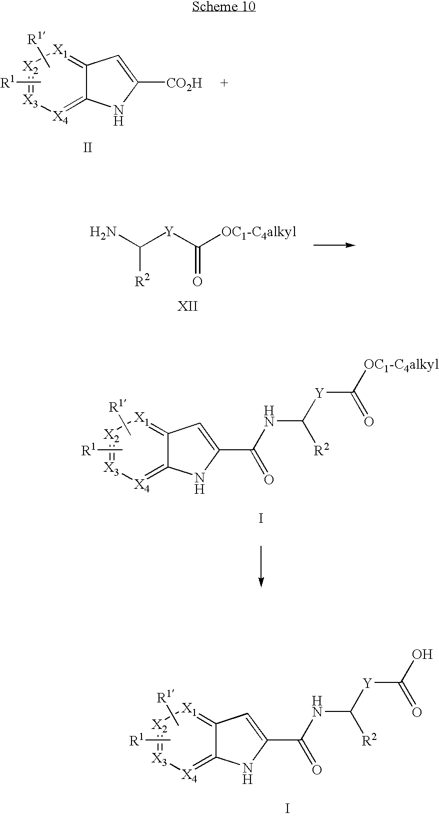

Compounds of Formula (I) (wherein Z is C═O and R³ is C₁₋₄alkoxy) may be prepared as illustrated in Scheme 10 by combination of compounds of Formula (II) and compounds of Formula (XII) under standard coupling conditions as described for Scheme 1. Compounds of Formula (XII) are generally commercially available or are readily prepared by known techniques

Compounds of Formula (I) (wherein Z is absent and R³ is —CO₂H) may be prepared by ester hydrolysis of compounds of Formula (I) (where Z is C═O and R³ is a C₁₋₄alkoxy group) using aqueous alkali typically at a temperature of around 25° C. for 30 min to 20 h.

During the synthesis of the compounds of Formula (I), labile functional groups in the intermediate compounds, e.g. hydroxy, carboxy and amino groups, may be protected. The compounds of Formula (II) may be protected in the 1-position e.g. with an arylmethyl, acyl, alkoxycarbonyl, sulfonyl or silyl group. The protecting groups may be removed at any stage in the synthesis of the compounds of Formula (I) or may be present on the final compound of Formula (I). A comprehensive discussion of the ways in which various labile functional groups may be protected and methods for cleaving the resulting protected derivatives is given in for example, Protective Groups in Organic Chemistry, T. W. Greene and P. G. M. Wuts, (1991) Wiley-Interscience, New York, 2^{nd} edition.

### EXPERIMENTAL

### Example 1

Synthesis of 5-chlorolpyrrolo[2,3-c]pyridine-2-carboxylic acid [1-(S)-4-fluorobenzyl)-2-(4-hydroxypiperidin-1-yl)-2-oxoethyl]amide hydrochloride
(a) Preparation of 5-chlorolyprrolo[2,3-c]pyridine-2-carbonyl chloride hydrochloride
Method A: 5-Chloropyrrolo[2,3-c]pyridine-2-carboxylic acid (39.3 g, 0.20 mol) was suspended in acetonitrile and heated to reflux. Thionyl chloride (44 mL, 71.4 g, 0.60 mol) was added dropwise over 20 min at reflux temperature. The resulting suspension was heated at reflux for a further 3 h (TLC monitoring: n-butanol-acetic acid-water 4:1:1, UV visualised. Sample was prepared by quenching into methanolic NH₃ solution). The reaction mixture was evaporated to dryness under reduced pressure and the crude product used in the next step without further purification. Yield 49.3 g (98.0%).
Method B: A slurry of 5-chloro-1H-pyrrolo[2,3-c]-pyridin-2-carboxylic acid (300 g, 1.52 mol) in acetonitrile (3.75L) was heated to reflux. Thionyl chloride (363 g, 3.052 mol, 223 mL) was added dropwise to the mixture and the reaction monitored by tlc and hlpc. After completion of the reaction excess thionyl chloride and acetonitrile was distilled off under diminished pressure to obtain a thick slurry. Toluene (2L) was added to the residue, and solvents evaporated under diminished pressure. The product was filtered off under nitrogen and washed with toluene (0.2L) and hexane (0.2L). The product was dried in vacuo at 45-50° C. over potassium hydroxide to obtain the title compound. Yield 368 g (96%). IR (KBr) 1750 cm⁻¹ (also 2436 br, 1981, 1869, 1631, 1588, 1529, 1447, 1389, 1340, 1289, 1203, 1140 and 1001 cm⁻¹).
(b) Preparation of N-(5-chloropyrrolo[2,3-c]pyridin-2-carbonyl)-L-4-fluorophenylalanine hydrochloride
Method A: To a solution of NaOH (9.41 g, 0.235 mol, 1.2 eq) and Na₂CO₃ (62.3 g, 0.588 mol, 3.0 eq) in deionized water (240 mL) was added L4-fluorophenylalanine (43.1 g, 0.235 mol, 1.2 eq) followed by THF (240 mL). The resulting solution was cooled to 0-5° C. and a suspension of 5-chloropyrrolo[2,3-c]pyridine-2-carbonyl chloride hydrochloride (49.3 g, 0.196 mol, 1.0 eq) in dry THF was added (˜30 min). The reaction mixture was stirred at 0-5° C. for 15 min (HPLC monitoring, direct analysis of the sample). The temperature was maintained at 0-5° C. while the pH of the reaction mixture was adjusted to ˜7 by the addition of conc. hydrochloric acid and THF was removed under reduced pressure. EtOAc (50 mL) was added to the remaining aqueous solution and the pH adjusted to 1-2 by the addition of conc. hydrochloric acid (˜80 mL altogether). The resulting suspension was stirred for 30 min at 0-5° C. The precipitate was then filtered, washed with EtOAc (2×100 mL) and dried in vacuo at 40° C. Crude yield: 67.6 g (86.6%). The crude product was crystallised from a mixture of 2M HCl (540 ml) and 2-propanol (270 ml). Yield 60.9 g (78.0%). ¹H-NMR (DMSO): 13.02 (br s, 1H), 9.2 (d, 1H), 8.80 (s, 1H), 7.95 (s, 1H), 7.48 (s, 1H), 7.34 (dd, 2H), 6.96 (dd, 2H), 4.81 (m,1H), 3.29 (dd, 1H), 3.16 (dd, 1H).
Method B: To a solution of NaOH (73.0 g, 1.82 mol) and Na₂CO₃ (486 g, 4.58 mol) in deionized water (1.90L) was added L-4-fluorophenylalanine (336 g, 1.82 mol) followed by THF (2.80L). The resulting solution was cooled to 0-5° C. and a suspension of 5-chloropyrrolo[2,3-c]pyridine-2-carbonyl chloride hydrochloride (383 g, 1.52 mol) in dry THF was added (˜30 min). The reaction mixture was stirred at 0-5° C. for 30 min (HPLC monitoring, direct analysis of the sample). The temperature was maintained at 0-5° C. while the pH of the reaction mixture was adjusted to ˜7 by the addition of conc. hydrochloric acid (230 mL) and THF was removed under reduced pressure. EtOAc (3.0 L) was added to the residue and the pH adjusted to 1-2 by the addition of conc. hydrochloric acid (0.6L). The resulting slurry was stirred for 30 min at 0-5° C. The precipitate was then filtered, washed with EtOAc (2×500 mL) and dried in vacuo at 40-50° C. (95% purity by HPLC).
(c) Preparation of 5-chloropyrrolo[2,3-c]pyridine-2-carboxylic acid [1-(S )-4-fluorobenzyl)2-(4-hydroxypiperidin-1-yl)2-oxoethyl]amide hydrochloride
Method A: N-5-Chloropyrrolo[2,3-c]pyridine-2-carbonyl)-L-4-fluorophenylalanine hydrochloride (60.9 g, 0.153 mol) was suspended in dry THF (460 mL) and the mixture was stirred at room temperature. 4-Hydroxypiperidine (35.7 g, 0.353 mol) was added portionwise (slight exotherm) and the mixture stirred at room temperature for 10 min. 4-(4,6-Dimethoxy-1,3,5-triazin-2-yl)4-methylmorpholinium chloride (51.2 g, 0.185 mol, prepared according to the method of Kunishima et al, Tetrahedron Letters, 1999, 40, 5327-5330) was then added in one portion. The reaction mixture was stirred at room temperature for 1 h (HPLC monitoring, direct sample analysis). The solvent was removed under reduced pressure and the residue partitioned between EtOAc (500 mL) and saturated Na₂CO₃ solution (500 mL)-water (600 mL) mixture. The organic layer was separated and the aqueous layer extracted with EtOAc (2×150 mL), the combined organic layers was washed with brine, dried over Na₂SO₄ and evaporated. Crude yield (base) 70.9 g. The crude product was crystallised from a mixture of 2M HCl (420 mL) and 2-propanol (210 mL) to give 35.1 g (47.7%) of a light yellow crystalline material (water content 13.2% and >98% optical purity). A second crystallisation from the same mixture gave 21.4 g (29.1%) pure product with >99% optical purity. ¹H-NMR (DMSO): 13.2 (br s, 1H), 9.24 (dd, 1H), 8.90 (s, 1H), 7.95 (s, 1H), 7.50 (s, 1H), 7.28 (dd, 2H), 6.96 (dd, 2H), 5.25 (qa, 1H), 3.12 (m, 1H), 1.85-1.115 (m, 9H).
Method B: N-(5-Chloropyrrolo[2,3-c]pyridine-2-carbonyl)-L,4-fluorophenylalanine hydrochloride (450 g, 1.13 mol) was suspended in dry THF (3.40L) and the mixture cooled to 20-25° C. 4-Hydroxypiperidine (264 g, 2.60 mol) was added portionwise (slight exotherm) and the mixture stirred at 20-25° C. for 5-10 min. 4-(4,6-Dimethoxy-1,3,5-triazin-2-yl)4methylmorpholinium chloride (380 g, 1.37 mol, prepared according to the method of Kunishima et al, Tetrahedron Letters, 1999, 40, 5327-5330) was then added. The reaction mixture was stirred at 20-25° C. (HPLC monitoring, direct sample analysis). The reaction mixture was poured into a stirred solution of sodium carbonate (700 g) in deionised water (7L), EtOAc (500 mL) was added and the mixture stirred for 10 min. The organic layer was separated and the aqueous layer extracted with EtOAc (1×1L and 1×500 mL), the combined organic layers was washed with brine (2.0L) and dried over Na₂SO₄ (70 g) and activated carbon (15 g) overnight before the solvent was evaporated. The crude product was dissolved in methanol (2.0L) and 2M HCl (2.50L), Celite (10 g) and activated carbon (10 g) added. The resulting slurry was stirred for 30 min. The mixture was filtered and the methanol removed under reduced pressure. The crystal slurry was cooled overnight to 4-5° C., filtered, washed with 2M HCl (0.20L) and dried in vacuo at 50° C. The product was recrystallised from a mixture of 2M HCl (2.10L) and 2-propanol (0.9L) and the product dried over KOH in vacuo at 50° C.

### Example 2

Recrystalisation of 5-chloropyrrolo[2,3-c]pyridine-2-carboxylic acid [1-(S)-4-fluorobenzyl)-2-(4-hydroxypiperidin-1-yl)-2-oxoethyl]amide hydrochloride from methanol:acetonitrile

5-Chloropyrrolo[2,3-c]pyridine-2-carboxylic acid [1-(S)-4-fluorobenzyl)-2-(4-hydroxypiperidin-1-yl)-2-oxoethyl]amide hydrochloride (10 g, material obtained according to Example 1 but without recrystallisation) was dissolved in methanol (20 mL) at 50° C. and under continuous stirring acetonitrile (100 mL) was added. The product started to precipitate at the end of the addition of acetonitrile. The mixture was warmed to 40° C. to get homogenous solution. After addition of the acetonitrile the suspension was cooled to 0° C. under continuous stirring. The product was crystallized for 12 h at 0° C. The precipitate was filtered on a sintered glass filter. The filter cake was washed with of acetonitrile (10 mL) and the product was dried at 45° C. in vacuum yielding product with >99% optical purity. Mp 77-78° C. Water content 4.5-5.5% w/w.

### Example 3

Preparation of 5-chloro-1H-pyrrolo[2,3-c]pyridine-2-carboxylic acid [1-(S)-(4-fluorobenzyl)-2-(4-hydroxypiperidin-1-yl)-2-oxoethyl]amide
Method A: To a solution of 2-(S)-[(5-chloro-1H-pyrrolo[2,3-c]pyridine-2-carbonyl)amino]-3-(4-fluorophenyl)propionic acid (1.4 g, 3.87 mmol) in DMF (35 mL) was added HATU (1.77 g, 4.64 mmol) and the reaction stirred for 10 min. 4-Hydroxypiperidine (0.43 g, 4.26 mmol) was added, followed by DIPEA (0.8 mL, 4.64 mmol,) and the reaction stirred at rt for 16 h. Solvent was removed in vacuo and the crude material partitioned between ethyl acetate (50 mL) and water (50 mL). The organics were washed with sodium bicarbonate (2×30 mL) and brine (2×30 mL), dried (MgSO₄) and the solvent removed in vacuo. Purification by column chromatography (SiO₂, 96:4 dichloromethane/methanol) gave the title compound. m/z (ES⁺)=445.15 [M+H]⁺; RT=3.24 min.
Method B: The title compound was prepared from 5-chloro-1H-pyrrolo[2,3-c]pyridine-2-carboxylic acid and 2-(S)-amino-3-(4-fluorophenyl)-1-(4-hydroxypiperidin-1-yl)propan-1-one hydrochloride. The product was purified by chromatography on silica gel eluting with methanol/dichloromethane (1:19) to give the title compound as a pale yellow solid. δ_{H} (CD₃OD): 1.08-1.19 (0.5H, m), 1.29-1.51 (1.5H, m), 1.54-1.62 (0.5H, m), 1.73-1.84 (1.5H, m), 3.06-3.36 (4H, m), 3.67-3.95 (2.5H, m), 4.03-4.10 (0.5H, m), 5.32 (1H, t), 6.97-7.04 (2H, m), 7.14 (1H, s), 7.26-7.33 (2H, m), 7.66 (1H, s), 8.55 (1H, s); m/z (ES⁺)=445 [M+H]⁺; RT=3.27 min.
The biological activity of the glycogen phosphorylase inhibitors for use in the method of the invention may be tested as follows:

### Glycogen Phosphorylase Assay In Vitro:

### Materials

α-D-Glucose-1-phosphate (disodium salt), Glycogen, D-Glucose, Malachite Green Hydrochloride, Ammonium Molybdate tetrahydrate, BSA, HEPES and rabbit muscle phosphorylase α (P1261) were purchased from Sigma. All other reagents were analytical grade.

### Method

An assay for glycogen phosphorylase activity in the reverse direction was developed based on the method described by Engers et al., Can. J Biochem., 1970, 48, 746-754]. Rabbit muscle glycogen phosphorylase α (Sigma) was reconstituted at a stock concentration of 100 μg/mL in 25 mM Tris/HCl. The pH was measured in a 96-well plate in a final volume of 100 μL containing 50 mM Hepes pH 7.2, 7.5 mM glucose, 0.5 mM glucose-1-phosphate and 1 mg/mL glycogen. After incubation at 30° C. for 30 min, the inorganic phosphate released from glucose-1-phosphate was measured by the addition of 150 μL of malachite green/molybdate solution prepared as follows: 5 mL of 4.2% ammonium molybdate in 4N HCl, 15 mL of 0.045% malachite green, 50 μL of Tween 20. Following a 30 min incubation at room temperature, the absorbance was measured at 620 nm. For IC₅₀ determination, 10 μL of a serial dilution of compound (100 μm to 0.004 μM) in DMSO was added to each reaction in duplicate with the equivalent concentration of DMSO added to the control uninhibited reaction. Dose response curves were then obtained by plotting % inhibition versus log₁₀ compound concentration. IC₅₀ is defined as the concentration of compound achieving 50% inhibition under the assay conditions described.

The compounds of the examples demonstrated activity as glycogen phosphorylase inhibitors in this assay.

The glycogen phosphorylase inhibitors for use in the method of the invention preferably have a measured IC₅₀ of lower than 100 μM. It is still more advantageous for the IC₅₀ to be lower than 50 μM. It is even more advantageous for the IC₅₀ to be lower than 5 μM. It is yet more advantageous for the IC₅₀ to be lower than 0.5 μM.

### In Vivo Animal Model to Mimic Human Night Time Dosing:

Diabetic ZDF male rats (Charles River) were allowed free access to tap water and enriched pelleted chow diet (M-Z Ereich; Art. No. V1185-000; Ssniff R Spezialdiäten GmbH, D-59494, Soest Germany) ad-libitum for 4 weeks until they were 8 weeks old. The animals were housed under a 16 hr/8 hr:dark/light phase (lights on 09:00). As the animals progressed to the diabetic state, weekly blood glucose and insulin measurement were made at the end of the light period (17:00 h) and a blood glucose measurement made at the end of the active (dark) cycle (08:30-09:00 h). When the animals had reached 8 weeks of age, they were trained on a meal paradigm, by removal of food during the light period (09:00-17:00 h), which was maintained for the remainder of the study. After the animals had reached 12 weeks of age, and their diabetic status had been confirmed using the fasted glucose measurements (>8 mM), the animals were divided into groups. Animal groups were sorted by body weight, blood glucose and plasma insulin concentrations to minimize inter group variation. Rats were dosed at 09:00 h with either vehicle (10% Gelucire 44/14; 90% water) or the compound of Example 3 (in Gelucire vehicle) via gavage using a feeding tube (15 g, 75 mm; Fine Science Tools, Heidelberg, Germany) at 4 weekly intervals when the animals were 12, 13, 14 and 15 weeks old. Blood glucose and insulin concentrations were measured by the glucose oxidase method (Super G Glucose analyzer; Dr. Müller Gerätebau, Freital, Germany) and Elisa technique, respectively, at T=−45 min, before dosing (08:15 h) and then at T=0 min (09:45 h), T=45 min (09:45 h), T=90 min (10:30 h), T=150 min (11:20 h) and T=360 min (15:00 h) after each weekly successive test compound dose.

The compound of Example 3 at 30 mg/kg po via gavage in 10% Gelucire 44/14 formulation resulted in a reduction of 6.28+/−2.47 mM (mean+/−S.D.) in blood glucose concentration; p=0.019 versus controls at 360 min after dosing at 14 weeks old, versus vehicle controls which showed a fall of only 2.79+/−1.81 mM (mean+/−S.D.) from the start of dosing (T=0).

What is claimed is:

1. A method of treatment of diabetes, or a diabetes related condition, comprising night time dosing of an inhibitor of glycogen phosphorylase.

2. The method according to claim 1, comprising night time dosing of an inhibitor of glycogen phosphorylase and administration of another anti-diabetic therapy.

3. The method according to claim 1, comprising night time dosing of an inhibitor of glycogen phosphorylase and administration of another anti-diabetic therapy in the day time.

4. The method according to claim 1 for the treatment of type II diabetes.

5. The method according to claim 1 wherein the glycogen phosphorylase inhibitor is administered to a subject after the subject has consumed their last meal of the day.

6. The method according to claim 1 wherein the glycogen phosphorylase inhibitor is administered at bedtime.

7. The method according to claim 1 wherein the glycogen phosphorylase inhibitor is administered once during a 24 hour period.

8. The method according to claim 2 wherein the other anti-diabetic therapy is administered once, twice or three times a day.

9. The method according to claim 2 wherein the other anti-diabetic is administered as a meal related or prandial treatment.

10. The method according to claim 2 wherein the other anti-diabetic agent is selected from PPAR agonists, biguanides, sulfonylureas and other insulin secretagogues, insulin sensitisers, alpha-glucosidase inhibitors, dipeptidyl peptidase IV inhibitors, glucokinase activators, GLP-1 and GLP-1 analogues, insulin and insulin analogues.

11. The method according to claim 10, wherein the alpha glucosidase inhibitor is selected from acarbose, emiglitate, miglitol and voglibose.

12. The method according to claim 11, wherein the alpha glucosidase inhibitor is acarbose.

13. The method according to claim 10, wherein the biguanide is selected from metformin, buformin and phenformin.

14. The method according to claim 13, wherein the biguanide is metformin.

15. The method according to claim 10, wherein the insulin secretagogue is selected from glibenclamide, glipizide, gliclazide, glimepiride, tolazamide and tolbutamide, acetohexamide, carbutamide, chlorpropamide, glibornuride, gliquidone, glisentide, glisolamide, glisoxepide, glyclopyamide, glycylamide, glipentide repaglinide and nateglinide.

16. The method according to claim 10, wherein the insulin sensitiser is a PPARy agonist insulin sensitiser.

17. The method according to claim 10, wherein the insulin sensitiser is selected from troglitazone, ciglitazone, pioglitazone, englitazone and rosiglitazone.

18. The method according to claim 1 wherein the glycogen phosphorylase inhibitor is 5-chloro-1H-pyrrolo[2,3-c]pyridine-2-carboxylic acid [1-(S)-(4-fluorobenzyl)-2-(4-hydroxypiperidin-1-yl)-2-oxoethyl]amide, or a pharmaceutically acceptable salt thereof.

19. The method according to claim 18 wherein the glycogen phosphorylase inhibitor is 5-chloro-1H-pyrrolo[2,3-c]pyridine-2-carboxylic acid [1-(S)-(4-fluorobenzyl)-2-(4-hydroxypiperidin-1-yl)-2-oxoethyl]amide hydrochloride.

20. The method according to claim 1 wherein the glycogen phosphorylase inhibitor is administered orally.
